(19) United States
(12) Patent Application Publication
(10) Pub. No.: US 2026/0259605 A1
(43) Pub. Date: Sep. 3, 2026

(54) SYSTEMS AND METHODS FOR BRAIN-COMPUTER INTERFACE AND CALIBRATING THE SAME

(71) Applicant: University of Pittsburgh - Of the Commonwealth System of Higher Education, Pittsburgh, PA (US)

(72) Inventors: Sudarshan SEKHAR, Pittsburgh, PA (US); Aaron P. BATISTA, Pittsburgh, PA (US); Patrick J. LOUGHLIN, Pittsburgh, PA (US)

(73) Assignee: University of Pittsburgh - Of the Commonwealth System of Higher Education, Pittsburgh, PA (US)

(21) Appl. No.: 18/716,911

(22) PCT Filed: Sep. 16, 2022
(86) PCT No.: PCT/US2022/043758

§ 371 Date: Jun. 5, 2024

Related U.S. Application Data
(60) Provisional application No. 63/286,963, filed on Dec. 7, 2021.

(51) Int. Cl. G06F 3/01; G06N 20/00

(52) U.S. Cl. CPC G06F 3/015; G06N 20/00

(57) ABSTRACT

Disclosed herein are embodiments of methods and systems for generating a control command to cause an action ac-cording to an incoming neural activity corresponding to an action intended by a subject. In one aspect, a plurality of calibrated decoders corresponding to a plurality of neural activities may be provided. Each of the plurality of calibrated decoders may be defined by a unique set of parameter values. In one aspect, an incoming neural activity can be applied to a mapping function to determine a decoder for the incoming neural activity. The decoder may have a set of parameter values different from those of the plurality of calibrated decoders. According to the set of parameter values, the decoder may generate a control command to cause the action.

### CROSS-REFERENCE TO RELATED APPLICATION

This application is related to and claims priority 35 U.S. § 119(e) to U.S. Provisional Application No. 63/286,963, filed Dec. 7, 2021, titled “SYSTEMS AND METHODS FOR BRAIN-COMPUTER INTERFACE AND CALIBRATING THE SAME,” the entire contents of which are incorporated herein by reference for all purposes.

### STATEMENT OF GOVERNMENT SUPPORT

This invention was made with government support under HD090125 awarded by the National Institutes of Health. The government has certain rights in the invention.

### FIELD OF DISCLOSURE

The present disclosure is generally related to use of neural signals, including but not limited to devices, systems and methods of determining decoders corresponding to the neural signals.

### BACKGROUND

The loss of (e.g., upper limb) motor function due to injury or disease affects the ability to perform physical activities of daily living, including operating electronic devices. Brain-computer interface (BCI) technology may provide a device control for use with computer-based applications. Device control that enables computer use or kinematic action can provide a means of connecting to the world or performing physical actions, and can greatly improve quality of life for those living with severe motor impairment.

### SUMMARY

Various embodiments disclosed herein are related to a method of calibrating a BCI. In some embodiments, the method includes determining, by at least one processor, neural activity of a subject corresponding to an intended movement. In some embodiments, the method includes applying, by the at least one processor in absence of using any kinematics information corresponding to the neural activity, a statistical test on the neural activity applied to each of a plurality of filter models, to identify a subset of the plurality of filter models that each passes the statistical test, each of the plurality of filter models defined by a unique set of parameter values. In some embodiments, the method includes determining, by the at least one processor using parameter values of the identified subset, a first set of parameter values to define a calibrated decoder for the neural activity.

In some embodiments, determining the neural activity includes receiving, via electrodes of a brain-computer interface, a plurality of channels of neural signals from the subject. In some embodiments, determining the neural activity includes reducing, by the at least one processor, the plurality of channels into a plurality of dimensions using a factor analysis. In some embodiments, determining the neural activity includes determining, by the at least one processor using supervised learning models, the neural activity corresponding to an intended action.

In some embodiments, the plurality of filter models include Kalman filters, and the statistical test includes a normalized innovation squared test. In some embodiments, the first set of parameter values includes at least one of: a value of Kalman parameter Q, a value of Kalman parameter C, or a value of Kalman parameter R.

In some embodiments, determining the first set of parameter values includes identifying, by the at least one processor from the parameter values of the identified subset, a first plurality of parameter values corresponding to a defined parameter. In some embodiments, determining the first set of parameter values includes forming, by the at least one processor, a histogram of the first plurality of parameter values. In some embodiments, determining the first set of parameter values includes forming, by the at least one processor, a normalized histogram by normalizing the histogram by a count of a parameter value with a highest count amongst the first plurality of parameter values. In some embodiments, the defined parameter comprises: Kalman parameter Q, Kalman parameter C, or Kalman parameter R. In some embodiments, the method includes identifying, by the at least one processor, parameter values from the normalized histogram that meet a defined threshold, and determining, by the at least one processor, an average of the identified parameter values, as a first parameter value of the first set of parameter values. In some embodiments, determining the average includes applying, by the at least one processor, a weight to each corresponding parameter value in the normalized histogram, the weight being a normalized count of the corresponding parameter, and determining, by the at least one processor, an average of the weighted parameter values.

Various embodiments disclosed herein are related to a system such as a BCI system. In some embodiments, the system includes at least one processor. In some embodiments, the at least one processor is configured to determine a neural activity of a subject corresponding to an intended movement. In some embodiments, the at least one processor is configured to apply, in absence of using any kinematics information corresponding to the neural activity, a statistical test on the neural activity applied to each of a plurality of filter models, to identify a subset of the plurality of filter models that each passes the statistical test, each of the plurality of filter models defined by a unique set of parameter values. In some embodiments, the at least one processor is configured to determine, using parameter values of the identified subset, a first set of parameter values to define a calibrated decoder for the neural activity.

In some embodiments, the at least one processor is configured to determine the neural activity by receiving, via electrodes of the system, a plurality of channels of neural signals from the subject. In some embodiments, the at least one processor is configured to determine the neural activity by reducing the plurality of channels into a plurality of dimensions using a factor analysis. In some embodiments, the at least one processor is configured to determine the neural activity by determining, using supervised learning models, the neural activity corresponding to an intended action.

In some embodiments, the plurality of filter models comprise Kalman filters, and the statistical test comprises a normalized innovation squared test. In some embodiments, the first set of parameter values comprises at least one of: a value of Kalman parameter Q, a value of Kalman parameter C, or a value of Kalman parameter R.

In some embodiments, the at least one processor is configured to determine the first set of parameter values by identifying, from the parameter values of the identified subset, a first plurality of parameter values corresponding to a defined parameter. In some embodiments, the at least one processor is configured to determine the first set of parameter values by forming a histogram of the first plurality of parameter values. In some embodiments, the at least one processor is configured to determine the first set of parameter values by forming a normalized histogram by normalizing the histogram by a count of a parameter value with a highest count amongst the first plurality of parameter values. In some embodiments, the defined parameter comprises: Kalman parameter Q, Kalman parameter C, or Kalman parameter R.

In some embodiments, the at least one processor is further configured to identify parameter values from the normalized histogram that meet a defined threshold; and determine an average of the identified parameter values, as a first parameter value of the first set of parameter values. In some embodiments, the at least one processor is configured to determine the average by: applying a weight to each corresponding parameter value in the normalized histogram, the weight being a normalized count of the corresponding parameter; and determining an average of the weighted parameter values.

Various embodiments disclosed herein are related to a method for BCI. In some embodiments, the method includes providing, by at least one processor, a plurality of calibrated decoders corresponding to a plurality of neural activities, each of the plurality of calibrated decoders defined by a unique set of parameter values. In some embodiments, the method includes applying, by the at least one processor, a mapping function on an incoming neural activity excluded from the plurality of neural activities, to determine a decoder for the incoming neural activity. In some embodiments, the decoder has a set of parameter values different from those of the plurality of calibrated decoders. In some embodiments, the incoming neural activity corresponds to an action intended by a subject.

In some embodiments, the mapping function is configured to provide interpolation between parameter values of a defined parameter for at least two calibrated decoders. In some embodiments, the method includes applying, by the at least one processor, the mapping function on another incoming neural activity that is included in the plurality of neural activities, to identify a corresponding calibrated decoder for the another incoming neural activity, from the plurality of calibrated decoders. In some embodiments, the method includes applying, by the at least one processor, the determined decoder to translate the incoming neural activity into a control command for the action intended by the subject.

In some embodiments, the mapping function incorporates a cubic spline that fits through parameter values of a defined parameter for at least two calibrated decoders. In some embodiments, the cubic spline fits through at least one other value determined according to each of the parameter values of the defined parameter. In some embodiments, the at least one other value includes at least one of: a first value that is a defined amount above a first of the parameter values of the defined parameter, and a second value that is the defined amount below a second of the parameter values of the defined parameter. In some embodiments, the at least one other value includes at least one of: a first value that is a defined amount above one of the parameter values and sign-inverted, and a second value that is the defined amount below the one of the parameter values and sign-inverted.

In some embodiments, the at least two calibrated decoders include a first decoder calibrated for a first neural activity of a first intended action, and a second decoder calibrated for a second neural activity of a second intended action. In some embodiments, the incoming neural activity has a metric that is intermediate between corresponding metrics of the first neural activity and the second neural activity.

Various embodiments disclosed herein are related to a BCI system. In some embodiments, the system includes at least one processor. In some embodiments, the at least one processor is configured to provide a plurality of calibrated decoders corresponding to a plurality of neural activities, each of the plurality of calibrated decoders defined by a unique set of parameter values. In some embodiments, the at least one processor is configured apply a mapping function on an incoming neural activity excluded from the plurality of neural activities, to determine a decoder for the incoming neural activity, the decoder having a set of parameter values different from those of the plurality of calibrated decoders. In some embodiments, the incoming neural activity corresponds to an action intended by a subject.

In some embodiments, the mapping function is configured to provide interpolation between parameter values of a defined parameter for at least two calibrated decoders. In some embodiments, the at least one processor is configured to apply the mapping function on another incoming neural activity that is included in the plurality of neural activities, to identify a corresponding calibrated decoder for the another incoming neural activity, from the plurality of calibrated decoders. In some embodiments, the at least one processor is configured to apply the determined decoder to translate the incoming neural activity into a control command for the action intended by the subject.

In some embodiments, the mapping function incorporates a cubic spline that fits through parameter values of a defined parameter for at least two calibrated decoders. In some embodiments, the cubic spline fits through at least one other value determined according to each of the parameter values of the defined parameter. In some embodiments, the at least one other value includes at least one of: a first value that is a defined amount above a first of the parameter values of the defined parameter, and a second value that is the defined amount below a second of the parameter values of the defined parameter. In some embodiments, the at least one other value includes at least one of: a first value that is a defined amount above one of the parameter values and sign-inverted, and a second value that is the defined amount below the one of the parameter values and sign-inverted.

In some embodiments, the at least two calibrated decoders comprise a first decoder calibrated for a first neural activity of a first intended action, and a second decoder calibrated for a second neural activity of a second intended action. In some embodiments, the incoming neural activity has a metric that is intermediate between corresponding metrics of the first neural activity and the second neural activity.

The disclosure further encompasses, in some aspects, a method for treatment of brain related disease, injury or disorder in a subject in need, including implementing any embodiment of BCI in the present disclosure, or using BCI according to any method in the present disclosure, in the subject. The disclosure further encompasses, in some aspects, a method for detection or diagnosis of brain damage, brain function disorder or brain injury in a subject in need, including implementing any embodiment of BCI in the present disclosure, or using a BCI according to any method in the present disclosure, in the subject.

The disclosure further encompasses, in some aspects, a method for control of an assistive device in a subject in need, including implementing any embodiment of BCI in the present disclosure, or using a BCI according to any method in the present disclosure, in the subject.

Both the foregoing summary and the following description of the drawings and detailed description are illustrative and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1 is a block diagram of a BCI system, according to one or more disclosed embodiments.

FIG. 2A is a diagram illustrating an example process of calibrating a BCI system, according to one implementation.

FIG. 2B is a diagram illustrating an example operation of the BCI system calibrated according to FIG. 2A, according to one implementation.

FIG. 3 is a diagram illustrating an example process of calibrating a BCI system, according to one or more disclosed embodiments.

FIG. 4 is a diagram illustrating an example process of selecting a decoder of a BCI system through a mapping function, according to one or more disclosed embodiments.

FIG. 5A is a plot illustrating a performance of a BCI system, according to one implementation.

FIG. 5B is a plot illustrating a performance of a BCI system, according to one or more disclosed embodiments.

FIG. 6 is a flow chart illustrating an example process of calibrating a BCI system, according to one or more disclosed embodiments.

FIG. 7 is a flow chart illustrating an example process of generating a command to perform an action through a BCI system, according to one or more disclosed embodiments.

FIG. 8 is a block diagram of a computing environment according to an example implementation of the present disclosure.

### DETAILED DESCRIPTION

Disclosed herein are related to calibration of a BCI system. In one aspect, a neural activity of a subject is recorded, while the subject imagines making different actions or movements. Statistical analyses can be performed on the recorded neural activity to determine one or more decoder parameters. The BCI system may construct or implement a decoder, according to the one or more decoder parameters. The decoder can generate a control command to cause an action (e.g., move a robotic arm) corresponding to the subject's intent or imagination.

Advantageously, the calibration of the BCI system can be performed in a prompt, dynamic and/or efficient manner. In one aspect, the calibration is performed without kinematics information corresponding to the neural activity, but is performed based on a neural activity corresponding to the subject's imagination, mental state or intent on performing an action. For example, the calibration can be performed based on a neural activity corresponding to the subject's imagination or intent on moving the robotic arm, in the absence of (or without requiring or involving) the subject observing an action of the robotic arm. Moreover, by performing calibration on the recorded neural activity corresponding to the subject's imagination, mental state or intent on performing different movements, multiple decoders can be calibrated promptly. In one aspect, different types of movements may be associated with corresponding decoders, and calibrating each decoder can involve a tedious and laborious process. For example, a first decoder may be utilized to cause a first type of action (e.g., a slow but meticulous movement) of a robotic arm, where a second decoder may be utilized to cause a second type of action (e.g., a ballistic movement or a wide range of movement) of the robotic arm. However, calibrating each decoder individually while the subject observes the action or movement in response to the subject's intent may be time consuming and burdensome to the subject. By performing calibration on the recorded neural activity corresponding to the subject's imagination or intent on performing different types of movements and then using a mapping function to learn an association between neural activity and a set of decoder parameters, multiple processes of calibrating different decoders for different types of movements can be obviated.

Disclosed herein are related to a BCI system that can determine a decoder from a plurality of decoders for generating a control command that can cause an action corresponding to an input neural activity. As discussed above, different types of movements may be associated with corresponding decoders. In one aspect, the BCI system includes a mapping function to select, identify or determine a decoder corresponding to an input neural activity. The mapping function may be obtained by interpolating two or more sets of parameter values of two or more decoders. The BCI system may apply the input neural activity to the mapping function to determine a set of parameter values of a decoder. In addition, the BCI system may in some embodiments generate a control command, according to the determined parameter values of the decoder, to render or cause an action corresponding to the neural activity.

Advantageously, by utilizing a mapping function, the BCI system can promptly/rapidly (e.g., within 100 ms) select, switch or change between different decoders, in response to a neural activity of the subject's imagination or intent to make different types of movements or a combination of different types of movements. Accordingly, the BCI system can support and/or cause a dynamic and wide range of movements or actions. Furthermore, by interpolating across/between decoder parameter values, the mapping function can be formed/obtained using a few sets of parameter values (or a few calibrated decoders), rather than a large number of sets of parameter values, such that the BCI system can be set or configured in an efficient manner.

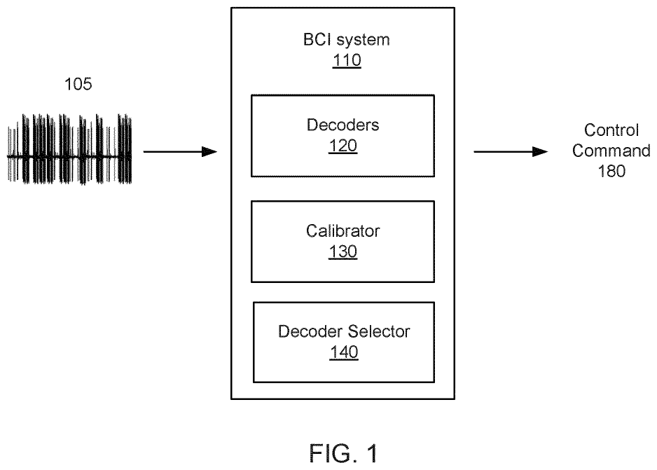

FIG. 1 is a block diagram of a BCI system 110, according to one or more disclosed embodiments. In one configuration, the BCI system 110 is communicatively coupled to a sensor including multiple electrodes that detect neural activity of a subject, and can generate input data 105 corresponding to the detected neural activity. The input data may be data representing sensor measurements of the neural activity. In one example configuration, the BCI system 110 is communicatively coupled to an actuator (e.g., a robot arm), and performs an action or a movement according to a control command 180. The control command 180 (e.g., control signal/output/response) may be data configuring or causing a corresponding action by the actuator. In this configuration, the BCI system 110 may receive the input data 105 corresponding to the neural activity of the subject corresponding to the subject's intent or imagination, and can generate the control command 180 according to the neural activity. In some embodiments, the BCI system 110 includes more, fewer, or different components than shown in FIG. 1. For example, the BCI system 110 may include the sensor including multiple electrodes. In some embodiments, the BCI system 110 may include or be communicatively coupled to the actuator.

In some embodiments, the BCI system 110 is embodied as a computing device. The BCI system 110 may include one or more processors and a memory (e.g., a non-transitory computer readable medium) storing instructions when executed by the one or more processors cause the one or more processors to perform one or more functions or processes described herein. In some embodiments, the BCI system 110 implements decoders 120, a calibrator 130, and a decoder selector 140. Each of the decoders 120, the calibrator 130, and/or the decoder selector 140 may be implemented as a hardware component, or a combination of a hardware component and a software component (e.g., program code executing on hardware).

In one aspect, the decoders 120 are components that generate the control command 180, for instance to cause or perform a corresponding (physical and/or virtual) action. Each decoder 120 may be formed and/or defined according to a corresponding set of parameter values. In one implementation, a decoder 120 may be implemented as a filter model or filter (e.g., Kalman filter) with a corresponding set of filter parameters. For example, a Kalman filter can be formed or configured with parameters including a state transition model (A), process noise (Q), observation model (C), and observation noise (R). In one aspect, different decoders 120 are configured with different parameter values and may be suited to generate the control command 180 to perform corresponding types of actions. For example, a first decoder 120 may generate the control command 180 to perform a first type of action (e.g., slow and meticulous movement of a robotic/virtual arm), where a second decoder 120 may generate the control command 180 to perform a second type of action (e.g., a ballistic movement or a wide range of movement of the robotic/virtual arm).

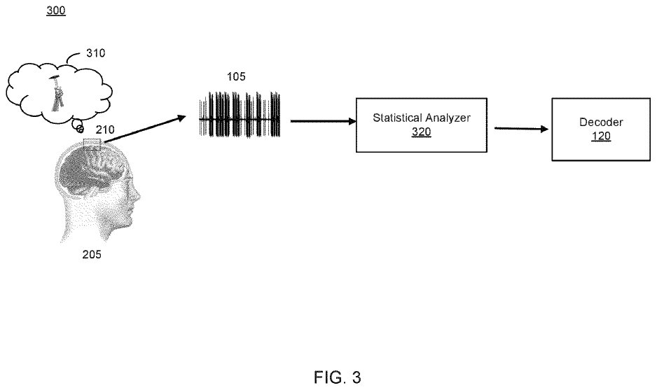

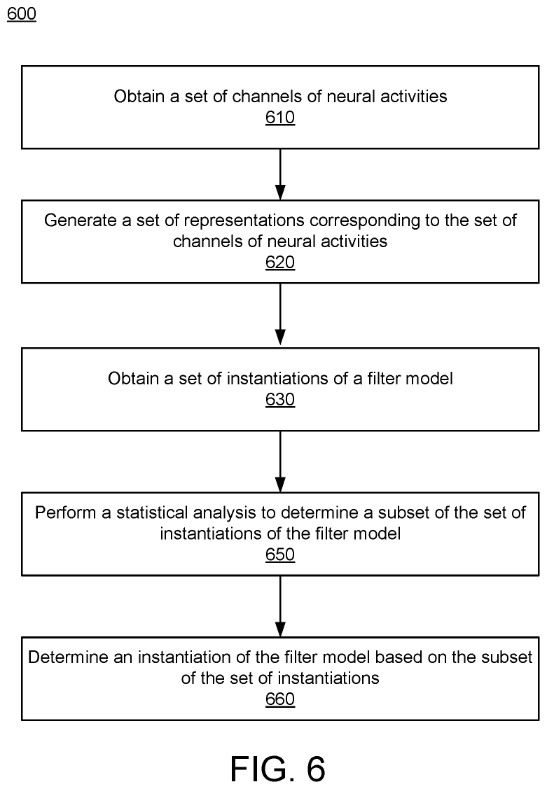

In one aspect, the calibrator 130 is a component that calibrates one or more decoders 120 to generate/output control commands 180 for instance. In one approach, the calibrator 130 may record a neural activity of a subject, while the subject imagines making/performing different actions or movements. For example, the subject may imagine causing a horizontal movement, a vertical movement, or a combination of these in a robotic/virtual/real arm with varying speeds. The calibrator 130 may perform statistical analyses or statistical tests to determine one or more calibrated decoder parameters. According to the one or more calibrated decoder parameters, a decoder 120 can be defined, constructed and/or implemented. Detailed descriptions on example calibration performed by the calibrator 130 are provided below with respect to FIGS. 3 and 6.

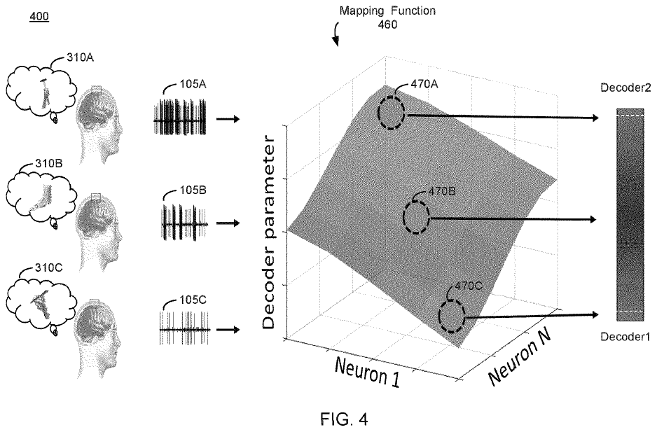

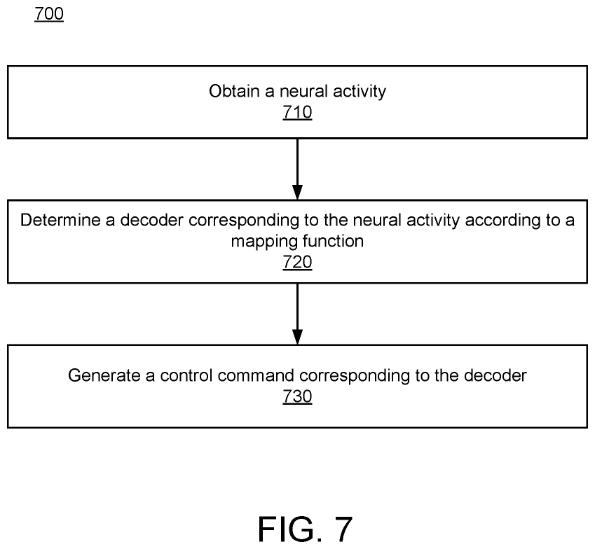

In one aspect, the decoder selector 140 is a component that selects/identifies/determines a decoder 120 from a range of decoders 120 for generating the control command 180 to perform or cause an action corresponding to the neural activity. In one aspect, the decoder selector 140 includes, implements, executes and/or stores a mapping function to select or determine a decoder 120 corresponding to an input neural activity. For example, the decoder selector 140 may apply the input data including N-channels of sensor measurements corresponding to the neural activity, to the mapping function to obtain a set of parameter values. For example, for a Kalman filter, the decoder selector 140 may determine a set of values of Q, C, and/or R parameters. According to the determined parameter values, the decoder 120 can be set or configured to generate the control command 180. Detailed descriptions on an example process of selecting/determining a decoder 120 are provided below with respect to FIGS. 4 and 7.

In one aspect, the decoder selector 140 may perform interpolation on two or more sets of parameter values of two or more decoders 120 to generate or obtain the mapping function. Although interpolation is sometimes referenced, such references are by way of illustration and not intended to be limiting in any way, and other calculation/estimation approaches can be incorporated (e.g., piecewise linear, polynomial fits, neural networks etc.). By interpolating decoder parameter values to generate the mapping function (which can extend to a large or infinite number of decoders), the decoder selector 140 can select/identify/determine, from a wide range of decoders 120, a decoder 120 to generate the control command 180 for causing an action corresponding to the neural activity, based on a few calibrated number of decoders 120 (or a few sets of calibrated parameter values of decoders 120).

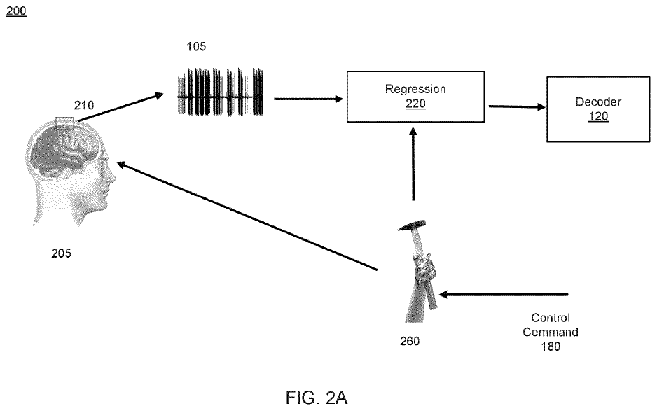

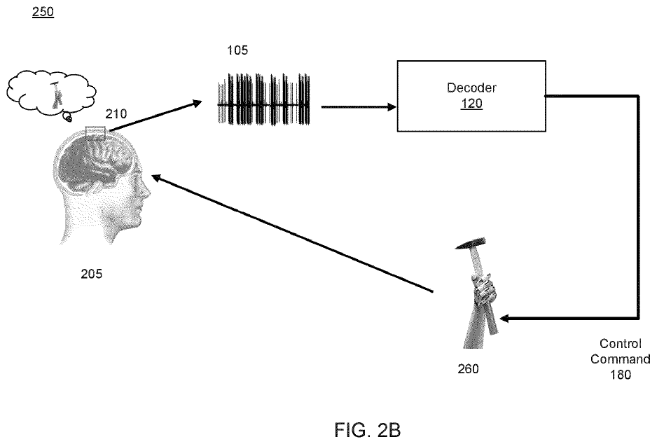

FIG. 2A is a diagram illustrating an example process 200 of calibrating a BCI system, according to one implementation. In one implementation, a robotic arm 260 may move or perform a swinging motion, in response to a control command 180 generated by a control device (not shown). The control device may be controlled or operated by a person performing calibration of the BCI system. A subject 205 may observe the movement or the swinging motion of the robotic arm 260. A sensor 210 including multiple electrodes may be mounted on or coupled to the subject's head (or brain) and can detect a neural activity generated in response to the user observing the movement or the swinging motion of the robotic arm 260. The sensor 210 may generate input data 105 representing sensor measurements of the neural activity. A computing device may receive the input data 105, and can apply the input data 105 to a regression model 220 to determine a set of parameter values of the decoder 120. The process 200 can be repeated to calibrate the BCI system.

FIG. 2B is a diagram illustrating an example operation 250 of the BCI system calibrated according to FIG. 2A, according to one implementation. After the decoder 120 is calibrated, the subject 205 may imagine causing a movement or swinging motion of the robotic arm 260. The sensor 210 may detect a neural activity corresponding to the user's imagination or intent, and generate input data 105 representing the sensor measurements of the neural activity. The decoder 120 may receive the input data 105 and generate the control command 180 corresponding to the detected neural activity. According to the control command 180, the robotic arm 260 may perform a movement or action corresponding to the user's imagination or intent.

Although the process 200 can be implemented to calibrate the decoder 120, the process 200 is performed while the subject 205 observes the movement or action of the robotic arm 260. Meanwhile, different types of movements may be associated with corresponding decoders 120, and calibrating different decoders 120 while the subject observes the actions or movements of the robotic arm 260 can be time consuming and tedious.

FIG. 3 is a diagram illustrating an example (e.g., improved) process 300 of calibrating the BCI system 110, according to one or more disclosed embodiments. In some embodiments, the decoder 120 is determined/trained/calibrated in absence of using any kinematics information corresponding to the neural activity. For example, the calibrator 130 may implement the statistical analyzer 320 that performs a statistical analysis or a statistical test on the neural activity represented by the input data 105, and can determine a set of calibrated parameter values of the decoder 120.

In one approach, the calibrator 130 receives/determines a neural activity of a subject corresponding to an intended movement. In one aspect, the calibrator 130 may receive, via electrodes of the sensor 210, the input data 105 represented by a plurality of channels (e.g., 50, 96 or other number of channels) of neural signals of the subject 205. Each channel may provide a corresponding dimension of information. The calibrator 130 may for instance perform a factor analysis or principal component analysis on the input data 105 to reduce a number of dimensions. For example, the calibrator 130 may generate or determine 10 dimensions of representations that are sufficient to convey or represent a significant portion of the neural activity. The calibrator 130 may also obtain, from the reduced number of dimensions of neural activity, a one-dimensional (or other concise/sufficient/accurate form of) representation of the neural activity. For example, the calibrator 130 may apply the 10 dimensions of the neural activity to neural-network/machine-learning and/or statistical analysis, such as a support vector machine (SVM) or a linear discriminant analysis (LDA), to obtain a one dimensional representation of the neural activity. The one dimensional representation may for example correspond to an intended action in a one dimensional space (e.g., horizontal movement or vertical movement). An intended action may comprise a planned, meant, deliberate, intentional, calculated and/or conscious action of a subject, or an action that is willed, caused, triggered and/or controlled by the subject's thought(s), neural activity or mental state.

In one approach, the calibrator 130 applies a statistical analysis or a statistical test (or other analysis/test) on the neural activity applied to each of a set of filter models to identify a subset of the set of filter models. Each filter model may be defined by a unique set of parameter values. For example, a Kalman filter may implement/model/represent the decoder 120, where each Kalman filter is defined, configured, or set, according to a corresponding set of Q, C, R parameter values. In one aspect, because the neural activity is reduced to one dimension, each parameter value (e.g., Q, C, R, A parameter value) can be scalar for instance. The A parameter can represent temporal correlation of kinematics at subsequent time points. And since kinematics may be decoded at short intervals or closely spaced time points (e.g., 50 ms), the temporal correlation can be set to approximately 1, such as 0.95 for instance. The calibrator 130 may obtain a range of parameter values of filter models, and can perform a statistical analysis on the filter models. Examples of the statistical analysis include a normalized innovation squared (NIS) test (related to covariance matching). Each filter model may be associated with a corresponding decoder 120. The calibrator 130 may determine filter models that satisfy or meet the statistical analysis to identify a subset of the set of filter models. For example, the calibrator 130 may obtain/identify 2500 filter models from over 16000 filter models.

In one approach, the calibrator 130 determines, using parameter values of the identified subset, a set of parameter values for defining, configuring, or setting a calibrated decoder 120. The calibrator 130 may identify, from the parameter values of the identified subset of the set of filter models, a plurality of parameter values (e.g., for a parameter such as Q, C or R), and can form a histogram of the plurality of parameter values. The calibrator 130 may form a normalized histogram by normalizing the histogram by factor, such as a count of a parameter value with a highest count amongst the plurality of parameter values, and can identify parameter values from the normalized histogram that meet a defined threshold (e.g., 0.5 or other value). Then, the calibrator 130 may determine an average of the identified parameter values, as a parameter value of one (e.g., Q, C or R) of the set of parameter values (for a filter model or decoder 120 corresponding to the neural activity). The calibrator 130 may determine the average by for instance applying a weight to each corresponding parameter value (e.g., that meet the defined threshold) in the normalized histogram. The weight may be based on a count of the corresponding parameter (according to or in the histogram), and can for instance be a normalized count of the corresponding parameter (e.g., from the normalized histogram). The calibrator 130 may determine an average of the weighted parameter values as a parameter value for the decoder 120 (corresponding to the neural activity).

FIG. 4 is a diagram illustrating an example process 400 of selecting a decoder 120 of the BCI system 110 through a mapping function 460, according to one or more disclosed embodiments. In some embodiments, the decoder selector 140 implements the mapping function 460 to select/identify/form/establish a decoder 120 from a range of decoders 120.

In one example approach, the decoder selector 140 may perform an interpolation on a few sets of parameter values (e.g., 470A, 470C) to obtain the mapping function 460. Examples of interpolation applied to obtain the mapping function 460 include a cubic spline, piecewise linear function, polynomial fits, or other interpolation/spline function. For example, the decoder selector 140 may obtain a median value of neural activity corresponding to a large amplitude of a one-dimensional movement. Then, two additional points with respect to the median value can be obtained. For example, a first point may be higher than the median value by a defined amount (e.g., 10%), and a second point may be lower than the median value by the defined amount. Assuming that the median value is 50, additional neural points e.g., 45 and 55 can be obtained. Then sign-inverted values of the above values can be obtained (e.g., −45, −50 & −55). The similar process can be performed for neural activity corresponding to a small amplitude of a one-dimensional movement. Assuming that the median value is 10, additional neural points (e.g., 15 and 5) can be obtained, and sign inverted values of the above values can be obtained (e.g., −15, −10, −5). The decoder selector 140 may perform interpolation by fitting a cubic spline to express/represent/model the Kalman parameters associated with neural activities as a function of the neural points mentioned above.

The decoder selector 140 may apply the input neural activity to the mapping function 460 to determine a set of parameter values of a decoder 120. For example, the decoder selector 140 may apply N-channel of input data 105A corresponding to intent 310A to the mapping function 460 to obtain a set 470A of parameter values to configure a decoder 120, e.g., that can be used to cause/model/decode a ballistic movement (e.g., swinging a hammer). For example, the decoder selector 140 may apply N-channel of input data 105C corresponding to intent 310C to the mapping function 460 to obtain a set 470C of parameter values to configure a decoder 120 that can be used to cause/model/decode a meticulous movement (e.g., writing with a pen). For example, the decoder selector 140 may apply N-channel of input data 105B corresponding to intent 310B to the mapping function 460 to obtain a set 470B of parameter values to configure a decoder 120 to cause/model/decode an intermediate movement (e.g., a movement that is intermediate between a ballistic movement and a meticulous movement, such as moving a cup).

By utilizing the mapping function 460, the decoder selector 140 can rapidly/dynamically/promptly (e.g., within 100 ms) select or change different decoders 120, in response to a neural activity of the subject's imagination or intent to make different types of movements or a combination of different types of movements. Accordingly, the decoder selector 140 can promptly establish, change or select different decoders 120 to cause/model/decode a dynamic and wide range of movements or actions. Furthermore, by interpolating decoder parameter values, the mapping function 460 can be obtained with a low number of sets (e.g., 470A, 470C) of parameter values, rather than a large number of sets of parameter values, such that the mapping function 460 can be obtained in an efficient manner.

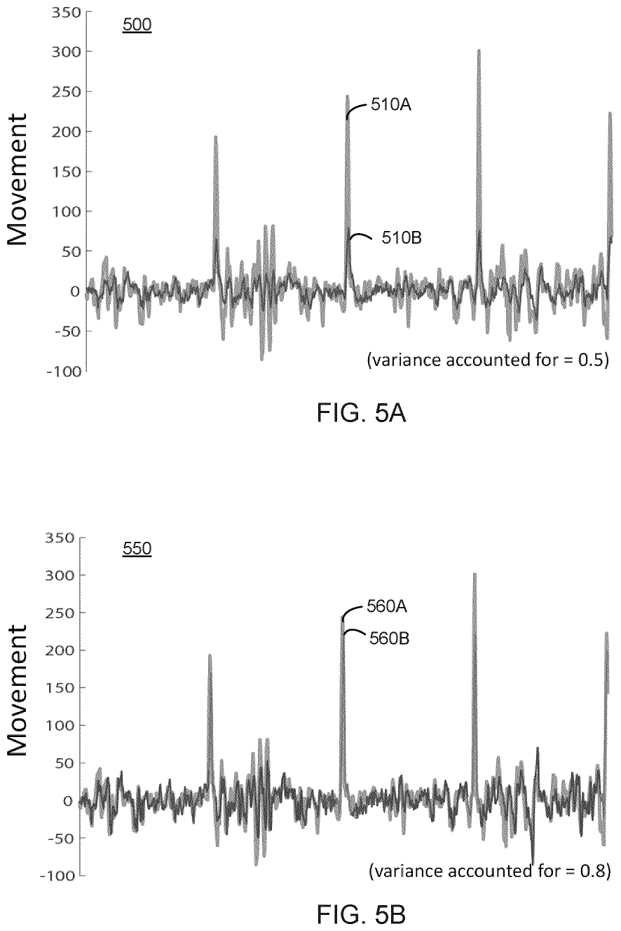

FIG. 5A is a plot 500 illustrating a performance of a BCI system with the calibration performed according to the process 200 for a small movement. FIG. 5B is a plot 550 illustrating a performance of the BCI system 110 with the calibration performed according to the process 300. In the plots 500, 550, an X-axis corresponds to a time and a Y-axis corresponds to a velocity of a movement (or hand velocity). Although the velocity of the movement is utilized to measure the performance of the BCI system 110 in FIGS. 5A and 5B, other characteristics (e.g., position or acceleration) can be utilized to measure the performance of the BCI system 110.

In the plot 500, a graph 510A corresponds to a recorded movement, and a graph 510B corresponds to a decoded movement. The movement may correspond to or may be representative of a cursor movement on a computer screen, or a physical movement of the robotic arm 260 performed/controlled by the decoder 120 calibrated as described in FIG. 2A. As shown in the plot 500, the graph 510B can track the graph 510A for small amplitudes of movements (e.g., between −25 mm/s and 25 mm/s), but the graph 510B does not follow or track the graph 510A well for large amplitudes/amounts of movements. Hence, the BCI system calibrated for a small movement according to the process 200 may not perform well to cause/model/decode a large movement.

In the plot 550, a graph 560A corresponds to a recorded movement, and a graph 560B corresponds to a decoded movement. The movement may correspond to or may be representative of a cursor movement on a computer screen, or a physical movement of the robotic arm 260 performed by the decoder 120 calibrated as described in FIG. 3. As shown in the plot 550, the graph 560B can track the graph 560A for a wide range of amplitudes (e.g., between −100 and 200). Hence, the BCI system 110 calibrated based on the statistical analysis according to the process 300 may perform well to provide/model/decode a wide range of movements.

FIG. 6 is a flow chart illustrating an example process 600 of calibrating the BCI system 110, according to one or more disclosed embodiments. In some embodiments, the process 600 is performed by the BCI system 110. In some embodiments, the process 600 is performed by a different entity. In some embodiments, the process 600 includes more, fewer, or different steps than shown in FIG. 6.

In one approach, the BCI system 110 receives/obtains 610 a set of channels of neural activities or neural signals. The BCI system 110 may obtain input data 105 corresponding to a set of channels of neural activities or neural signals via/from a sensor including multiple electrodes, where each electrode is associated with a corresponding channel. In one example, the sensor may obtain 96 channels of neural activities or neural signals. Neural activity can be collected from the motor cortex of subjects (e.g., Rhesus monkeys or humans) using 96-electrode arrays for instance.

In one approach, the BCI system 110 obtains, generates or determines 620 a set of representations corresponding to the set of channels of neural activities or neural signals. The number of representations may be smaller than a number of channels. The BCI system 110 may for example perform a factor analysis or principal component analysis on the input data 105 to reduce a number of dimensions. For example, the BCI system 110 may determine 10 dimensions of representations that are sufficient/relevant to convey or represent a significant portion of 96 channels of the neural activity. For example, the 10 dimension of representations may be a weighted combination of 96 channels of the neural activity. The BCI system 110 may also obtain, from the reduced number of dimensions of neural activity, a one dimensional representation of the neural activity. For example, the BCI system 110 may apply the 10 dimensions of the neural activity to an artificial intelligence based or other model/analysis, e.g., a support vector machine (SVM) or a linear discriminant analysis (LDA), to obtain a one dimensional representation of the neural activity. The one dimensional representation may correspond to an intended/actual action in a one dimensional space (e.g., horizontal movement or vertical movement). In one aspect, the SVM can be implemented to find a linear classification boundary that separates neural activity along a one dimensional movement (e.g., leftward movement and rightward movement). The decision boundary may be a 9-dimensional (9D) hyperplane for instance. The vector orthogonal to this hyperplane may represent the direction in a 10-dimensional (10D) neural space along which neural activity relevant to a one-dimensional movement is encoded. The 10-dimensional neural activity during a one-dimensional movement may be projected to this vector. Projection of neural activity to this vector may be/provide the single dimensional representation of neural activity encoding horizontal movements.

In one approach, the BCI system 110 obtains 630 a set of instantiations of a decoder 120 (e.g., having a common set of parameters of various values). An instantiation of a decoder 120 may be set, defined or configured, according to a set of parameter values. Assuming for an example that a Kalman filter is implemented to set, define or configure a decoder 120, when the neural activity is reduced to one dimension, each parameter value (e.g., Q, C, R parameter value) can be a scaler value. In one example, the state-transition model A can be set to a predetermined value (e.g., 0.95), because the state-transition model (A) represents the correlation between the velocity at time t-1 (Vt-1) and at time t (Vt). Because the BCI system 110 can operate on the order of milliseconds, Vt-1 and Vt may be highly correlated, and the state-transition model A close to 1 may be a reasonable approximation. In one example, a wide range of Q, C & R can be sampled. For example, Q ranging from ~80 to ~8000, R ranging from ~80 to ~800, and C ranging from ~0.2 to ~0.5 can be obtained, such that 16000 (or other numbers of) unique combinations of Q, C & R, each representing a different instantiation of the Kalman filter, can be obtained.

In one example approach, the BCI system 110 performs 650 a statistical analysis or statistical tests to determine a subset of the set of instantiations of a filter model. In one example, a set of neural activities recorded during performance of one-dimensional movement is selected. The BCI system 110 may perform a statistical analysis on the set of neural activities and each of the 16000 unique instantiations of the Kalman filter. Examples of the statistical analysis include NIS test (related to covariance matching techniques). In one aspect, the NIS test is implemented to check if a set of Kalman parameters (A, Q, C, R) are optimal (appropriate) for the latent state being estimated (e.g., velocity, acceleration and/or distance of a one-dimensional movement). In one aspect, the NIS test is implemented to check which of the 16000 unique instantiations of the Kalman filter are statistically appropriate/possible/candidates for the set of neural data obtained for one-dimensional movement. The subset of the set of instantiations of the Kalman filters can be determined by determining or identifying the subset of the set of instantiations that pass the NIS-test. For example, 2500 of instantiations can be determined/identified/obtained from 16000 instantiations.

In one example approach, the BCI system 110 determines 660 an instantiation of the filter model based on the subset of the set of instantiations. In one example, the BCI system 110 constructs a marginal histogram for each of Kalman parameter (e.g., Q, C & R parameter). Each of the three histograms (one for Q, C & R respectively) may be normalized by the frequency of their respective maximum occurring values. Kalman parameters with a normalized frequency less than a threshold value (e.g., 0.5) may be discarded for example. The BCI system 110 may determine the final values of each Kalman parameter Q, C & R by averaging the (e.g., non-discarded) Kalman parameter values in each histogram, where the averages may be weighted by the normalized frequency for instance.

FIG. 7 is a flow chart illustrating an example process 700 of generating a command to perform an action through the BCI system 110, according to one or more disclosed embodiments. In some embodiments, the process 700 is performed by the BCI system 110. In some embodiments, the process 700 is performed by other entities. In some embodiments, the process 700 includes more, fewer, or different steps than shown in FIG. 7.

In one approach, the BCI system 110 obtains 710 a neural activity. For example, the BCI system 110 may obtain input data 105 corresponding to a neural activity from a sensor 210. The BCI system 110 may obtain a neural activity recorded during performance of a one-dimensional movement.

In one approach, the BCI system 110 selects 720 (e.g., determines, defines and/or establishes) a decoder 120 corresponding to the neural activity. For example, the BCI system 110 may apply the neural activity to the mapping function 460 to determine a corresponding decoder 120. The mapping function 460 can be obtained/implemented by extending from (e.g., performing interpolation on) a few sets of parameter values (e.g., 470A, 470C). Examples of interpolation applied to obtain the mapping function 460 include a cubic spline, a polynomial spline, piecewise linear function or other spline or interpolation function.

In one example approach/embodiment, the BCI system 110 generates 730 a control command 180 corresponding to the selected/determined decoder 120. The BCI system 110 may set, define or configure the decoder 120 according to a set of parameter values corresponding to the neural activity from the mapping function. The decoder 120 set, defined or configured according to the set of parameter values can generate/output the control command 180, e.g., which can be used to cause an intended action using an actuator for instance.

Advantageously, by utilizing a mapping function, the BCI system 110 can promptly (e.g., within 100 ms) select or change different decoders 120, in response to a neural activity of the subject's imagination or intent to make/perform different types of movements or a combination of different types of movements. Accordingly, the BCI system 110 can cause/model/decode a dynamic and wide range of movements or actions, e.g., to be performed by the actuator (e.g., robotic arm) for instance. Furthermore, by interpolating decoder parameter values, the mapping function can be obtained with a fewer/low number of sets of parameter values, rather than a large number of sets of parameter values, such that the BCI system 110 can be set or configured in an efficient manner.

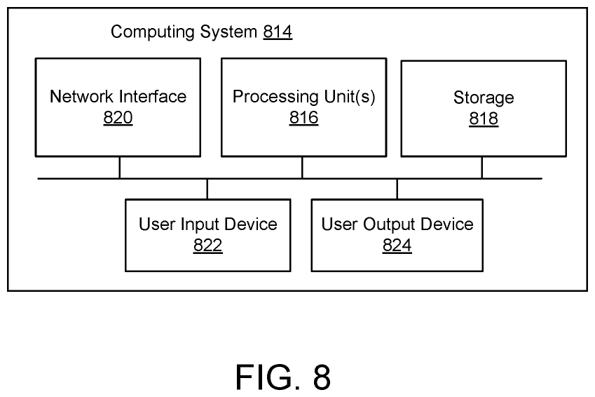

Various operations described herein can be implemented on computer systems. FIG. 8 shows a block diagram of a representative computing system 814 usable to implement the present disclosure. In some embodiments, the BCI system 110 is implemented by the computing system 814. Computing system 814 can be implemented, for example, as a consumer device such as a smartphone, other mobile phone, tablet computer, wearable computing device (e.g., smart watch, eyeglasses, head mounted display), desktop computer, laptop computer, cloud computing service or implemented with distributed computing devices. In some embodiments, the computing system 814 can include computer components such as processors 816, storage device 818, network interface 820, user input device 822, and user output device 824.

Network interface 820 can provide a connection to a wide area network (e.g., the Internet) to which WAN interface of a remote server system is also connected. Network interface 820 can include a wired interface (e.g., Ethernet) and/or a wireless interface implementing various RF data communication standards such as Wi-Fi, Bluetooth, or cellular data network standards (e.g., 3G, 4G, 5G, 60 GHz, LTE, etc.).

User input device 822 can include any device (or devices) via which a user can provide signals to computing system 814; computing system 814 can interpret the signals as indicative of particular user requests or information. User input device 822 can include any or all of a keyboard, touch pad, touch screen, mouse or other pointing device, scroll wheel, click wheel, dial, button, switch, keypad, microphone, sensors (e.g., a motion sensor, an eye tracking sensor, etc.), and so on.

User output device 824 can include any device via which computing system 814 can provide information to a user. For example, user output device 824 can include a display to display images generated by or delivered to computing system 814. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). A device such as a touchscreen that function as both input and output device can be used. Output devices 824 can be provided in addition to or instead of a display. Examples include indicator lights, speakers, tactile “display” devices, printers, and so on.

Some implementations include electronic components, such as microprocessors, storage and memory that store computer program instructions in a non-transitory computer readable storage medium. Many of the features described in this specification can be implemented as processes that are specified as a set of program instructions encoded on a computer readable storage medium. When these program instructions are executed by one or more processors, they cause the processors to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processor 816 can provide various functionality for computing system 814, including any of the functionality described herein as being performed by a server or client, or other functionality associated with message management services.

It will be appreciated that computing system 814 is illustrative and that variations and modifications are possible. Computer systems used in connection with the present disclosure can have other capabilities not specifically described here. Further, while computing system 814 is described with reference to particular blocks, it is to be understood that these blocks are defined for convenience of description and are not intended to imply a particular physical arrangement of component parts. For instance, different blocks can be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Implementations of the present disclosure can be realized in a variety of apparatus including electronic devices implemented using any combination of circuitry and software.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed in connection with one implementation are not intended to be excluded from a similar role in other implementations or implementations.

The hardware and data processing components used to implement the various processes, operations, illustrative logics, logical blocks, modules and circuits described in connection with the embodiments disclosed herein may be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device, etc.) may include one or more devices (e.g., RAM, ROM, Flash memory, hard disk storage, etc.) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an example embodiment, the memory is communicably connected to the processor via a processing circuit and includes computer code for executing (e.g., by the processing circuit and/or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or embodiment, and references to “an implementation,” “some implementations,” “one implementation” or the like are not necessarily mutually exclusive and are intended to indicate that a particular feature, structure, or characteristic described in connection with the implementation can be included in at least one implementation or embodiment. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements. Technical and scientific terms used herein have the meanings commonly understood by one of ordinary skill in the art, unless otherwise defined. Any suitable materials and/or methodologies known to those of ordinary skill in the art can be utilized in carrying out the methods described herein.

Systems and methods described herein may be embodied in other specific forms without departing from the characteristics thereof. As used herein, “approximately,” “about” “substantially” or other terms of degree will be understood by persons of ordinary skill in the art and will vary to some extent on the context in which it is used. If there are uses of the term which are not clear to persons of ordinary skill in the art given the context in which it is used, references to “approximately,” “about” “substantially” or other terms of degree shall include variations of +/−10% from the given measurement, unit, or range unless explicitly indicated otherwise.

Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining may be stationary (e.g., permanent or fixed) or moveable (e.g., removable or releasable). Such joining may be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References to “or” can be construed as inclusive so that any terms described using “or” can indicate any of a single, more than one, and all of the described terms. A reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

References herein to the positions of elements (e.g., “top,” “bottom,” “above,” “below”) are merely used to describe the orientation of various elements in the FIGURES. The orientation of various elements may differ according to other example embodiments, and that such variations are intended to be encompassed by the present disclosure.

As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A method comprising:
determining, by at least one processor, a neural activity of a subject corresponding to an intended movement;
applying, by the at least one processor in absence of using any kinematics information corresponding to the neural activity, a statistical test on the neural activity applied to each of a plurality of filter models, to identify a subset of the plurality of filter models that each passes the statistical test, each of the plurality of filter models defined by a unique set of parameter values; and
determining, by the at least one processor using parameter values of the identified subset, a first set of parameter values to define a calibrated decoder for the neural activity.

2. The method of claim 1, wherein determining the neural activity comprises:
receiving, via electrodes of a brain-computer interface, a plurality of channels of neural signals from the subject;
reducing, by the at least one processor, the plurality of channels into a plurality of dimensions using a factor analysis; and
determining, by the at least one processor using supervised learning models, the neural activity corresponding to the intended movement.

3. The method of claim 1, wherein the plurality of filter models comprise Kalman filters, and the statistical test comprises a normalized innovation squared test.

4. The method of claim 1, wherein the first set of parameter values comprises at least one of: a value of Kalman parameter Q, a value of Kalman parameter C, or a value of Kalman parameter R.

5. The method of claim 1, wherein determining the first set of parameter values comprises:
identifying, by the at least one processor from the parameter values of the identified subset, a first plurality of parameter values corresponding to a defined parameter;
forming, by the at least one processor, a histogram of the first plurality of parameter values; and
forming, by the at least one processor, a normalized histogram by normalizing the histogram by a count of a parameter value with a highest count amongst the first plurality of parameter values.

6. The method of claim 5, wherein the defined parameter comprises: Kalman parameter Q, Kalman parameter C, or Kalman parameter R.

7. The method of claim 5, further comprising:
identifying, by the at least one processor, parameter values from the normalized histogram that meet a defined threshold; and
determining, by the at least one processor, an average of the identified parameter values, as a first parameter value of the first set of parameter values.

8. The method of claim 7, wherein determining the average comprises:
applying, by the at least one processor, a weight to each corresponding parameter value in the normalized histogram, the weight being a normalized count of the corresponding parameter; and
determining, by the at least one processor, an average of the weighted parameter values.

9. A system comprising:
at least one processor configured to:
determine a neural activity of a subject corresponding to an intended movement,
apply, in absence of using any kinematics information corresponding to the neural activity, a statistical test on the neural activity applied to each of a plurality of filter models, to identify a subset of the plurality of filter models that each passes the statistical test, each of the plurality of filter models defined by a unique set of parameter values, and
determine, using parameter values of the identified subset, a first set of parameter values to define a calibrated decoder for the neural activity.

10. The system of claim 9, wherein the at least one processor is configured to determine the neural activity by:
receiving, via electrodes of the system, a plurality of channels of neural signals from the subject;
reducing the plurality of channels into a plurality of dimensions using a factor analysis; and
determining, using supervised learning models, the neural activity corresponding to an intended action.

11. The system of claim 9, wherein the plurality of filter models comprise Kalman filters, and the statistical test comprises a normalized innovation squared test.

12. The system of claim 9, wherein the first set of parameter values comprises at least one of: a value of Kalman parameter Q, a value of Kalman parameter C, or a value of Kalman parameter R.

13. The system of claim 9, wherein the at least one processor is configured to determine the first set of parameter values by:
identifying, from the parameter values of the identified subset, a first plurality of parameter values corresponding to a defined parameter;
forming a histogram of the first plurality of parameter values; and
forming a normalized histogram by normalizing the histogram by a count of a parameter value with a highest count amongst the first plurality of parameter values.

14. The system of claim 13, wherein the defined parameter comprises: Kalman parameter Q, Kalman parameter C, or Kalman parameter R.

15. The system of claim 13, wherein the at least one processor is further configured to:
identify parameter values from the normalized histogram that meet a defined threshold; and
determine an average of the identified parameter values, as a first parameter value of the first set of parameter values.

16. The system of claim 15, wherein the at least one processor is configured to determine the average by:
applying a weight to each corresponding parameter value in the normalized histogram, the weight being a normalized count of the corresponding parameter; and
determining an average of the weighted parameter values.

17. A method comprising:
providing, by at least one processor, a plurality of calibrated decoders corresponding to a plurality of neural activities, each of the plurality of calibrated decoders defined by a unique set of parameter values; and
applying, by the at least one processor, a mapping function on an incoming neural activity excluded from the plurality of neural activities, to determine a decoder for the incoming neural activity, the decoder having a set of parameter values different from those of the plurality of calibrated decoders,
wherein the incoming neural activity corresponds to an action intended by a subject.

18. The method of claim 17, wherein the mapping function is configured to provide interpolation between parameter values of a defined parameter for at least two calibrated decoders.

19. The method of claim 17, comprising:
applying, by the at least one processor, the mapping function on another incoming neural activity that is included in the plurality of neural activities, to identify a corresponding calibrated decoder for the another incoming neural activity, from the plurality of calibrated decoders.

20. The method of claim 17, comprising:
applying, by the at least one processor, the determined decoder to translate the incoming neural activity into a control command for the action intended by the subject.

21-42. (canceled)
